(19) United States
(12) Patent Application Publication
(10) Pub. No.: US 2024/0212838 A1
(43) Pub. Date: Jun. 27, 2024

(54) PREDICTING MENTAL STATE CHARACTERISTICS OF USERS OF WEARABLE DEVICES

(71) Applicant: Hewlett-Packard Development Company, L.P., Spring, TX (US)

(72) Inventors: Jishang Wei, Guilford, CT (US); Rafael Antonio Ballagas, Palo Alto, CA (US); Erika H. Siegel, Palo Alto, CA (US)

(21) Appl. No.: 18/557,768

(22) PCT Filed: Apr. 29, 2021
(86) PCT No.: PCT/US2021/029857

§ 371 Date: Oct. 27, 2023

(51) Int. Cl. G16H 40/63; A61B 5/00; A61B 5/16; G06F 1/16; G06N 5/04

(52) U.S. Cl. CPC G16H 40/63; A61B 5/165; A61B 5/6803; G06F 1/163; G06N 5/04

(57) ABSTRACT

An example method includes generating. with sensors of a wearable device. a plurality of physiological measures of a user of the wearable device while the user is performing a task. The method includes processing. with an inference engine of the wearable device. the plurality of physiological measures. The method includes generating. with the inference engine. a task difficulty class prediction and a residual estimation based on the processed physiological measures. The method includes generating, with the inference engine. a predicted value of a current mental state characteristic of the user based on the task difficulty class prediction and the residual estimation.

### Background

Augmented reality (AR) systems and virtual reality (VR) systems may include a head-mounted display (HMD) that is tracked in a three-dimensional (3D) workspace. These systems allow the user to interact with a virtual world.

### BRIEF DESCRIPTION OF THE DRAWINGS

FIG. 1 is a block diagram illustrating elements of a wearable device according to an example.

FIG. 2 is a block diagram illustrating elements of an inference engine according to an example.

FIG. 3 is a diagram illustrating the sampling and labeling of physiological sensor data according to an example.

FIG. 4 is a flow diagram illustrating a method for predicting a current mental state characteristic of a user of a wearable device according to an example.

FIG. 5 is a block diagram illustrating a head mounted display according to an example.

FIG. 6 is a block diagram illustrating a non-transitory computer-readable storage medium according to an example.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

Some examples disclosed herein are directed to a virtual reality headset with sensors to sense a plurality of physiological characteristics (e.g., pupillometry, eye activity, heart activities, etc.) of the user, and a cognitive load inference engine that generates a class prediction and a residual estimation based on the sensed physiological characteristics. The class prediction represents a task difficulty (e.g., “low”, “medium”, or “high” difficulty) for a task being performed by the user. Each of the task difficulties may be associated with a typical cognitive load level. In some examples, the cognitive load levels associated with the task difficulties are average demanding cognitive load values for “low”, “medium”, and “high” difficulty tasks. The residual estimation may be a regression output that may be combined with the typical cognitive load level associated with the class prediction to generate a predicted value of a current cognitive load of the user. In some examples, the inference engine provides calibration-free, real-time and continual point estimates of a cognitive load currently being experienced by a user. “Cognitive load” as used in some examples disclosed herein refers to the amount of mental effort for a person to perform a task or learn something new.

The training for the inference engine may involve collecting sensor readings from a training group of users while they perform tasks, and receiving their subjective ratings of experienced cognitive load. The collected data may also include task difficulty information for tasks, including a typical cognitive load value associated with each task difficulty. The collected data may be processed using a sliding window to generate a plurality of signal samples with associated labels. A set of features may be identified for each of the signal samples. The features may be processed using representation learning neural networks to generate learned representations of the data. The learned representations may be fused together into a fused representation, which may be provided to a class prediction neural network and a residual estimation neural network for training. The inference engine may be trained using two targets: (1) a classification target of task difficulty of a task the user is performing (e.g., “low”, “medium”, or “high” difficulty); and (2) a regression target of cognitive load of the user relative to a typical value for the task difficulty (e.g., relative amount of cognitive load the user is experiencing for a specific task compared to the population-wide average cognitive load for performing that task).

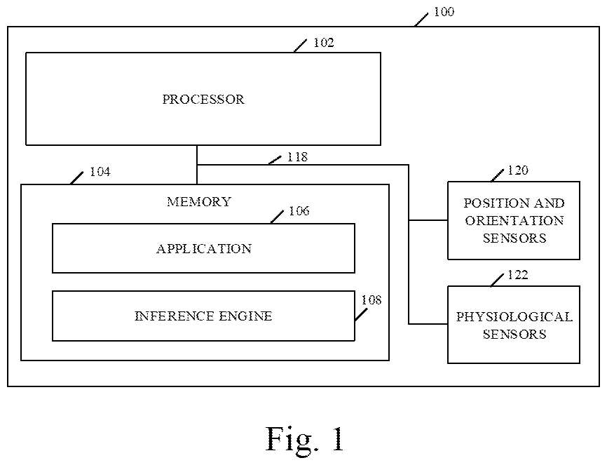

FIG. 1 is a block diagram illustrating elements of a wearable device 100 according to an example. In an example, wearable device 100 is a VR or AR headset or other head mounted display (HMD) device. Wearable device 100 includes at least one processor 102, memory 104, position and orientation sensors 120, and physiological sensors 122. In the illustrated example, processor 102, memory 104, and sensors 120 and 122 are communicatively coupled to each other via communication link 118.

Processor 102 includes a central processing unit (CPU) or another suitable processor. In one example, memory 104 stores machine readable instructions executed by processor 102 for operating the device 100. Memory 104 includes any suitable combination of volatile and/or non-volatile memory, such as combinations of Random Access Memory (RAM), Read-Only Memory (ROM), flash memory, and/or other suitable memory. These are examples of non-transitory computer readable storage media. The memory 104 is non-transitory in the sense that it does not encompass a transitory signal but instead is made up of at least one memory component to store machine executable instructions for performing techniques described herein.

Memory 104 stores application module 106 and inference engine module 108. Processor 102 executes instructions of modules 106 and 108 to perform some techniques described herein. Application module 106 generates a 3D visualization that is displayed by device 100. In an example, inference engine module 108 infers high-level insights about a user of device 100, such as cognitive load, emotion, stress, engagement, and health conditions, based on lower-level sensor data, such as that measured by physiological sensors 122. In an example, inference engine module 108 is based on a machine learning model that is trained with a training set of data to be able to predict a task difficulty class of a task being performed by a user, and a residual estimation representing a relative amount of cognitive load the user is experiencing during the task compared to, for example, a population-wide average cognitive load for performing that task. The inference engine module 108 may combine the residual estimation with an average demanding cognitive load value associated with the predicted task difficulty class to generate a predicted value of a current cognitive load of the user. It is noted that some or all of the functionality of modules 106 and 108 may be implemented using cloud computing resources.

The device 100 may implement stereoscopic images called stereograms to represent a 3D visualization. The 3D visualization may include still images or video images. The device 100 may present the 3D visualization to a user via a number of ocular screens. In an example, the ocular screens are placed in an eyeglass or goggle system allowing a user to view both ocular screens simultaneously. This creates the illusion of a 3D visualization using two individual ocular screens. The position and orientation sensors 120 may be used to detect the position and orientation of the device 100 in 3D space as the device 100 is positioned on the user's head, and the sensors 120 may provide this data to processor 102 such that movement of the device 100 as it sits on the user's head is translated into a change in the point of view within the 3D visualization.

Although one example uses a VR headset to present the 3D visualization, other types of environments may also be used. In an example, an AR environment may be used where aspects of the real world are viewable in a visual representation while a 3D object is being drawn within the AR environment. Thus, much like the VR system described herein, an AR system may include a visual presentation provided to a user via a computer screen or a headset including a number of screens, among other types of devices to present the 3D visualization. Thus, the present description contemplates the use of not only a VR environment but an AR environment as well. Techniques described herein may also be applied to other environments.

In some examples, physiological sensors 122 are implemented as a multimodal sensor system that includes a plurality of different types of sensors to sense or measure different physiological or behavioral features of a user wearing the device 100. In some examples, physiological sensors 122 include a first sensor to track a user's pupillometry, a second sensor to track eye activity of the user, and a third sensor to track heart activities of the user (e.g., a pulse photoplethysmography (PPG) sensor). In other examples, physiological sensors 122 may include other types of sensors, such as an electromyography (EMG) sensor. Device 100 may also receive and process sensor signals from sensors that are not incorporated into the device 100.

In one example, the various subcomponents or elements of the device 100 may be embodied in a plurality of different systems, where different modules may be grouped or distributed across the plurality of different systems. To achieve its desired functionality, device 100 may include various hardware components. Among these hardware components may be a number of processing devices, a number of data storage devices, a number of peripheral device adapters, and a number of network adapters. These hardware components may be interconnected through the use of a number of busses and/or network connections. The processing devices may include a hardware architecture to retrieve executable code from the data storage devices and execute the executable code. The executable code may, when executed by the processing devices, cause the processing devices to implement at least some of the functionality disclosed herein.

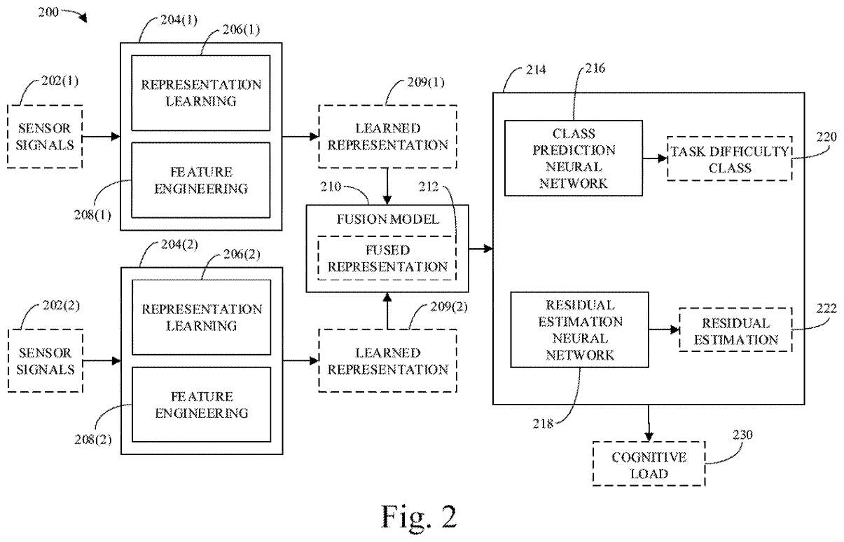

FIG. 2 is a block diagram illustrating elements of an inference engine 200 according to an example. In an example, inference engine module 108

(FIG. 1) is implemented with inference engine 200. Inference engine 200 includes a plurality of feature generation modules 204(1)-204(2) (collectively referred to as feature generation modules 204), a fusion model module 210, and a prediction module 214. The feature generation modules 204(1) and 204(2) include representation learning modules 206(1) and 206(2) (collectively referred to as representation learning modules 206), respectively, and feature engineering modules 208(1) and 208(2) (collectively referred to as feature engineering modules 208), respectively. Prediction module 214 includes class prediction neural network 216 and residual estimation neural network 218.

In some examples, inference engine 200 predicts users' cognitive loads in real-time while they are performing cognitively demanding tasks in VR environments. In a specific context, a person's mental efforts are a product of the demand of a task and the cognitive capacity when the person is performing the task. Since cognitive loads may be influenced by multiple factors, some examples involve training a machine learning model to predict “ground truth” cognitive loads using both people's subject cognitive load ratings and task difficulties as inference objectives. In this way, the model may be trained by exploring commonalities, differences, and regularization across both objectives.

For the training of inference engine 200, a plurality of different tasks in a VR environment may be designed, which involve different levels of mental effort (e.g., low, medium, and high) to complete. In an example, the medium difficulty task may be a multitasking task that completely includes the low difficulty task, and the high difficulty task may be a multitasking task that completely includes the medium difficulty task. For example, the low difficulty task may be a visual vigilance task; the medium difficulty task may be the visual vigilance task and an arithmetic task; and the high difficulty task may be the visual vigilance task, the arithmetic task, and an audio vigilance task. Thus, in this example, higher level tasks are objectively harder than lower level tasks.

A training group of people may be recruited to perform the tasks. While each participant is performing the tasks, physiological sensor signals for the participant may be collected, such as the participant's pupillometry, eye activity, and heart activity information. These sensor signals are each a temporal series of data and are represented in FIG. 2 by sensor signals 202(1)-202(2) (collectively referred to as sensor signals 202). For each individual task performed by each participant, the task difficulty level of the task may be recorded, and the participant may be asked after completion of the task to provide a subjective rating of the demanding cognitive load experienced by the participant during performance of the task. In an example, the subjective cognitive load experienced by the participant is a continuous value, c, falling in the range from 0 to 1, where 0 and 1 represent the lowest and highest experienced cognitive loads, respectively. In an example, for each task, each participant provides one subject cognitive load value for the entire task.

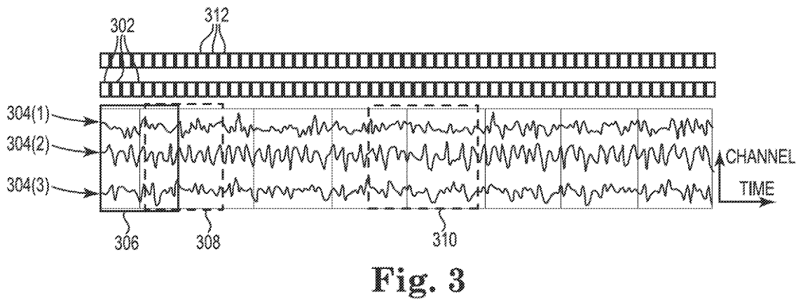

Thus, at this point in the training, physiological sensor signals 202 and labels (i.e., subjective ratings of cognitive load, and task difficulty levels) will have been collected for each task performed by each of the participants. A next step in the training process is to process the physiological sensor signals 202 and labels. FIG. 3 is a diagram illustrating the sampling and labeling of physiological sensor data according to an example. FIG. 3 shows simplified representations of a plurality of different types of physiological sensor signals 304(1)-304(3) (collectively referred to as sensor signals 304) over time for a single task performed by a single participant. Sensor signals 304 are an example of sensor signals 202 (FIG. 2). A sliding window 306 may be used to generate signal samples from the sensor signals 304. In an example, the sliding window 306 has a width of 12.5 seconds and is moved across the sensor signals 304 with a one second skip step. Thus, as the sliding window 306 is moved across the sensor signals 304, it will reach position 308 and then position 310, and then eventually reach the end of the sensor signals 304. In an example, signal samples may be obtained individually from each of the sensor signals 304. A demanding cognitive load label is associated with each of the signal samples, as represented by labels 302 positioned above the sensor signals 304. Each label 302 represents the subjective cognitive load value experienced by the participant while completing the task, which, in an example, is a continuous value, c, falling in the range from 0 to 1, where 0 and 1 represent the lowest and high experienced cognitive loads, respectively. A task difficulty label is also associated with each of the signal samples, as represented by labels 312. Each label 312 represents the task difficulty value for the task, which, in an example, is a discrete value, d, with the options of “low”, “medium”, and “high”

In an example, each of the feature engineering modules 208 (FIG. 2) is associated with one of the sensor signals 202 and generates the signal samples and labels (e.g., labels 302 and 312 shown in FIG. 3) for its associated sensor signals 202. Each of the feature engineering modules 208 then generates a set of predefined features from each of the signal samples of the sensor signals 202 associated with that feature engineering module 208. In an example, each set of features is represented as an n-dimensional vector, v_n, where n represents the number of features. Each set of features may include various statistical, temporal, and frequency domain features, such as pupil diameters, blink, saccade, fixation, heart rate statistics, heart rate variabilities, respiration rate, and power spectral densities for PPG signals, as well as other features.

The n-dimensional vectors representing the sets of features associated with sensor signals 202(1) are provided to representation learning module 206(1) to generate a learned representation 209(1) corresponding to the sensor signals 202(1). The n-dimensional vectors representing the sets of features associated with sensor signals 202(2) are provided to representation learning module 206(2) to generate a learned representation 209(2) corresponding to the sensor signals 202(2). Learned representations 209(1) and 209(2) may be collectively referred to as learned representations 209. Each of the learned representations 209 represents a high-level representation of the sensor signal modality associated with that representation 209. The representation learning modules 206 may generate the learned representations 209 using representation learning neural networks, such as convolutional neural networks (CNNs) to extract local dependency patterns from input sequences. In an example, each of the learned representations 209 is an m-dimensional vector, v_m, where m represents the dimensionality of the signal representation. The representations 209 may be generated through a model that is trained separately through unsupervised learning.

Fusion model module 210 fuses the learned representations 209 into a fused representation 212, which is provided to class prediction neural network 216 and residual estimation neural network 218. In an example, fusion model module 210 uses a CNN to facilitate the determination of the fused representation 212. In an example, the class prediction neural network 216 outputs a predicted task difficulty class 220 based on the fused representation 212 provided as an input and the residual estimation neural network 218 outputs a residual estimation 222 based on the fused representation 212 provided as an input.

In some examples, a typical demanding cognitive load value is determined for each of the possible task difficulties classes that might be output by class prediction neural network 216. For tasks with low/medium/high difficulty levels, the cognitive load values to be associated with these difficulty levels may be based on domain knowledge, e.g., [0.25, 0.5, 0.75], in a 0-1 range, or based on population-wide statistics. In some examples, the population average of reported subjective cognitive load ratings when people are completing a specific task may be used. The following are example values that may be used: (1) Task 1: Visual Vigilance, having a population average of subjective cognitive load rating of 0.240; (2) Task 2: Visual Vigilance+Arithmetic, having a population average of subjective cognitive load rating of 0.532; and (3) Task 3: Visual Vigilance+Arithmetic+Audio Vigilance, having a population average of subjective cognitive load rating of 0.728.

A relative subjective rating, c_r, may be calculated by subtracting the mean cognitive load, mean(d), of the corresponding task from the absolute subjective rating, c, as shown in the following Equation 1:

### Equation 1

c_r=c−mean(d)

This relative subjective rating, c_r, may be used as labels for the regression task performed by residual estimation neural network 218, while the task difficulty levels may be used as labels for the classification task performed by class prediction neural network 216. During training, all the task difficulty levels that cover the experienced cognitive load may be forced to estimate the correct target. During inference, the task difficulty level with maximum confidence may be selected as the predicted task difficulty class 220, and the final output, which is cognitive load value 230, may be computed by applying the estimated residual 222 to the average demanding cognitive load value associated with the predicted task difficulty class 220.

During training, the neural network weights from the representation learning modules 206 may be fixed, and the feature engineering modules 208 represent a set of deterministic algorithms/rules that have no weights to be tuned. During inference according to an example, inputs of multiple modalities (e.g., sensor signals 202) may be sent to the inference engine 200, which will continually output an updated cognitive load value 230 representing an estimate of the cognitive load currently being experienced by the user.

Various machine learning models may be used to predict cognitive load using features extracted from different signals, including k-nearest neighbor (KNN), naïve bayes (NB), logistic regression, linear discriminant analysis (LDA), support vector machine (SVM), ensemble methods (e.g., random forest and XGBoost), and neural networks. These machine learning models may be trained to predict a user's cognitive load levels (e.g., discrete values) based on physiological features from one or multiple signal modalities. In some examples, the machine leaning models may be trained with discrete cognitive load labels. In other examples, the machine learning models may be trained with both discrete and continuous labels, which can help the models to detect the “ground truth” cognitive loads. Some examples use a dual target scheme for cognitive load inference engine training and prediction. The two targets may be task demanding cognitive load and subjectively experienced cognitive load.

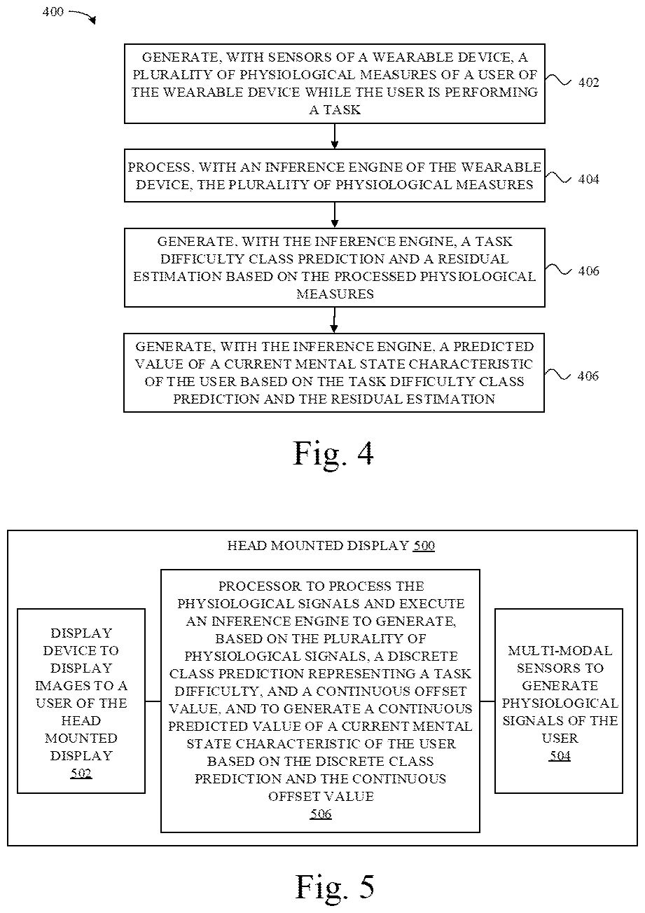

FIG. 4 is a flow diagram illustrating a method 400 for predicting a current mental state characteristic of a user of a wearable device according to an example. At 402, the method 400 includes generating, with sensors of a wearable device, a plurality of physiological measures of a user of the wearable device while the user is performing a task. At 404, the method 400 includes processing, with an inference engine of the wearable device, the plurality of physiological measures. At 406, the method 400 includes generating, with the inference engine, a task difficulty class prediction and a residual estimation based on the processed physiological measures. At 408, the method 400 includes generating, with the inference engine, a predicted value of a current mental state characteristic of the user based on the task difficulty class prediction and the residual estimation.

In method 400, the current mental state characteristic may be a current cognitive load of the user. The task difficulty class prediction may represent a discrete label for a difficulty level of the task the user is performing, and the residual estimation may be a continuous offset value. The method 400 may further include associating a mental state characteristic value with each of a plurality of task difficulty classes, wherein the task difficulty class prediction is selected from the plurality of task difficulty classes; and combining the residual estimation with the mental state characteristic value associated with the task difficulty class prediction to generate the predicted value of the current mental state characteristic of the user.

In method 400, the wearable device may be a head mounted display, and the sensors may be multi-modal and sense a plurality of different types of physiological measures of the user of the head mounted display. The physiological measures may include at least one of pupillometry information, eye activity information, and heart activity information.

In method 400, the processing may include: for each of the physiological measures, using a sliding window over time across the physiological measure to generate a plurality of signal samples corresponding to the physiological measure; for each of the physiological measures, extracting a set of features from each of the signal samples corresponding to the physiological measure; for each of the physiological measures, generating a learned representation corresponding to the physiological measure based on the set of features corresponding to the physiological measure; and fusing the learned representations for all of the physiological measures together to form a fused representation, and wherein the task difficulty class prediction and the residual estimation are generated with the inference engine based on the fused representation.

In method 400, the inference engine may be based on a trained machine learning model, wherein the method 400 further includes training the machine learning model, and wherein the training includes: generating a plurality of physiological measures of each of a plurality of test set users of wearable devices while the test set users perform tasks of varying difficulty; receiving, from each of the test set users for each of the tasks, a continuous subjective rating label for the mental state characteristic experienced by that test set user during that task; receiving a discrete objective difficulty label for each of the tasks performed by the test set users; and performing a multiple target learning process based on the physiological measures, the continuous subjective rating labels, and the discrete objective difficulty labels. The multiple target learning process may use a classification target of estimating task difficulty and a regression target of estimating a continuous value representing a relative level of the current mental state characteristic.

FIG. 5 is a block diagram illustrating a head mounted display 500 according to an example. The head mounted display 500 includes a display device 502 to display images to a user of the head mounted display. The head mounted display 500 includes multi-modal sensors 504 to generate physiological signals of the user. The head mounted display 500 includes a processor 506 to process the physiological signals and execute an inference engine to generate, based on the plurality of physiological signals, a discrete class prediction representing a task difficulty, and a continuous offset value, and to generate a continuous predicted value of a current mental state characteristic of the user based on the discrete class prediction and the continuous offset value.

The head mounted display 500 may be a virtual reality (VR) headset. The current mental state characteristic may be a current cognitive load of the user. In the head mounted display 500, a mean cognitive load value may be associated with each of a plurality of task difficulty classes, wherein the discrete class prediction may be selected from the plurality of task difficulty classes, and wherein the continuous offset value may be combined with the mean cognitive load value associated with the class prediction to generate the continuous predicted value of the current cognitive load of the user.

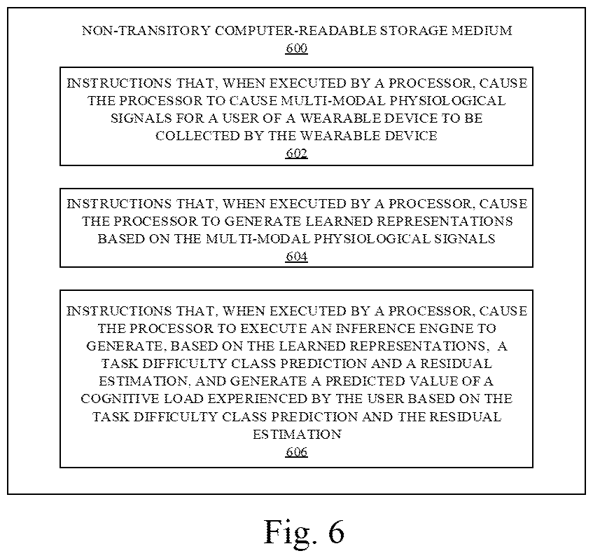

FIG. 6 is a block diagram illustrating a non-transitory computer-readable storage medium 600 according to an example. The non-transitory computer-readable storage medium 600 stores instructions 602 that, when executed by a processor, cause the processor to cause multi-modal physiological signals for a user of a wearable device to be collected by the wearable device. The non-transitory computer-readable storage medium 600 stores instructions 604 that, when executed by a processor, cause the processor to generate learned representations based on the multi-modal physiological signals. The non-transitory computer-readable storage medium 600 stores instructions 606 that, when executed by a processor, cause the processor to execute an inference engine to generate, based on the learned representations, a task difficulty class prediction and a residual estimation, and generate a predicted value of a cognitive load experienced by the user based on the task difficulty class prediction and the residual estimation.

For the non-transitory computer-readable storage medium 600, the task difficulty class prediction may represent a discrete label for a difficulty level of the task the user is performing, and the residual estimation may be a continuous offset value.

Although some examples disclosed herein may involve inferences related to cognitive load, other examples may involve other types of inferences, such as stress, engagement, emotion, and others, including quantizing a prediction uncertainty for such inferences.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. A method, comprising:
generating, with sensors of a wearable device, a plurality of physiological measures of a user of the wearable device while the user is performing a task;
processing, with an inference engine of the wearable device, the plurality of physiological measures;
generating, with the inference engine, a task difficulty class prediction and a residual estimation based on the processed physiological measures; and
generating, with the inference engine, a predicted value of a current mental state characteristic of the user based on the task difficulty class prediction and the residual estimation.

2. The method of claim 1, wherein the current mental state characteristic is a current cognitive load of the user.

3. The method of claim 1, wherein the task difficulty class prediction represents a discrete label for a difficulty level of the task the user is performing, and wherein the residual estimation is a continuous offset value. 4 The method of claim 1, wherein the method further comprises:
associating a mental state characteristic value with each of a plurality of task difficulty classes, wherein the task difficulty class prediction is selected from the plurality of task difficulty classes; and
combining the residual estimation with the mental state characteristic value associated with the task difficulty class prediction to generate the predicted value of the current mental state characteristic of the user.

5. The method of claim 1, wherein the wearable device is a head mounted display, and wherein the sensors are multi-modal and sense a plurality of different types of physiological measures of the user of the head mounted display.

6. The method of claim 1, wherein the physiological measures comprise at least one of pupillometry information, eye activity information, and heart activity information.

7. The method of claim 1, wherein the processing comprises:
for each of the physiological measures, using a sliding window over time across the physiological measure to generate a plurality of signal samples corresponding to the physiological measure;
for each of the physiological measures, extracting a set of features from each of the signal samples corresponding to the physiological measure;
for each of the physiological measures, generating a learned representation corresponding to the physiological measure based on the set of features corresponding to the physiological measure; and
fusing the learned representations for all of the physiological measures together to form a fused representation, and wherein the task difficulty class prediction and the residual estimation are generated with the inference engine based on the fused representation.

8. The method of claim 1, wherein the inference engine is based on a trained machine learning model, wherein the method further comprises training the machine learning model, and wherein the training comprises:
generating a plurality of physiological measures of each of a plurality of test set users of wearable devices while the test set users perform tasks of varying difficulty;
receiving, from each of the test set users for each of the tasks, a continuous subjective rating label for the mental state characteristic experienced by that test set user during that task;
receiving a discrete objective difficulty label for each of the tasks performed by the test set users; and
performing a multiple target learning process based on the physiological measures, the continuous subjective rating labels, and the discrete objective difficulty labels.

9. The method of claim 8, wherein the multiple target learning process uses a classification target of estimating task difficulty and a regression target of estimating a continuous value representing a relative level of the current mental state characteristic.

10. A head mounted display, comprising:
a display device to display images to a user of the head mounted display;
multi-modal sensors to generate physiological signals of the user; and
a processor to process the physiological signals and execute an inference engine to generate, based on the plurality of physiological signals, a discrete class prediction representing a task difficulty, and a continuous offset value, and to generate a continuous predicted value of a current mental state characteristic of the user based on the discrete class prediction and the continuous offset value.

11. The head mounted display of claim 10, wherein the head mounted display is a virtual reality (VR) headset.

12. The head mounted display of claim 10, wherein the current mental state characteristic is a current cognitive load of the user.

13. The head mounted display of claim 12, wherein a mean cognitive load value is associated with each of a plurality of task difficulty classes, wherein the discrete class prediction is selected from the plurality of task difficulty classes, and wherein the continuous offset value is combined with the mean cognitive load value associated with the class prediction to generate the continuous predicted value of the current cognitive load of the user.

14. A non-transitory computer-readable storage medium storing instructions that, when executed by a processor, cause the processor to:
cause multi-modal physiological signals for a user of a wearable device to be collected by the wearable device;
generate learned representations based on the multi-modal physiological signals; and
execute an inference engine to generate, based on the learned representations, a task difficulty class prediction and a residual estimation, and generate a predicted value of a cognitive load experienced by the user based on the task difficulty class prediction and the residual estimation.

15. The non-transitory computer-readable storage medium of claim 14, wherein the task difficulty class prediction represents a discrete label for a difficulty level of the task the user is performing, and wherein the residual estimation is a continuous offset value.
